(19) United States
(12) Patent Application Publication
(10) Pub. No.: US 2015/0135420 A1
(43) Pub. Date: May 21, 2015

(54) PROTECTIVE GEAR

(71) Applicant: Evo Consulting & Operations, LLC, Parker, CO (US)

(72) Inventor: Colin Daugherty, Lone Tree, CO (US)

(73) Assignee: Evo Consulting & Operations, LLC

(21) Appl. No.: 14/541,410

(22) Filed: Nov. 14, 2014

Related U.S. Application Data
(60) Provisional application No. 61/906,391, filed on Nov. 19, 2013.

(51) Int. Cl. A41D 13/05; A61F 9/02; F41H 1/02

(52) U.S. Cl. CPC A41D 13/0518; F41H 1/02; A61F 9/02
USPC 2/463; 2/2.5

(57) ABSTRACT

Protective gear is disclosed including a protective vest and eyewear. An example method of protecting a user both during and after an attack may include providing the protective vest to be worn by the user during the attack; and providing eyewear to obscure vision of the user from traumatic scenes following the attack.

### PRIORITY CLAIM

This application claims the priority benefit of U.S. Provisional Patent Application No. 61/906,391 filed Nov. 19, 2013 titled “Critical Incident Protective Gear” of Colin Daugherty, hereby incorporated by reference in its entirety as though fully set forth herein.

### BACKGROUND

In many instances, a person cannot predict when a critical incident or traumatic event may occur. Those people working at or attending environments and venues open to the public, such as schools, government agencies, and other facilities (e.g., office buildings or hospitals), may be at higher risk of being involved in a critical incident. Providing readily available protective gear, however, often encumbers the user physically (or emotionally—not wanting to unreasonably alarm children to potential threats), and therefore is often not even made available.

### BRIEF DESCRIPTION OF THE DRAWINGS

FIG. 1 is a perspective view of an example protective st shown with a ballistic barrier inserted therein.

FIG. 2 is a perspective view of the example protective vest shown in FIG. 1, as the protective vest may be worn by a person.

FIG. 3A is a front plan view of the protective vest; and FIG. 3B is a rear plan view of the protective vest.

FIG. 4A is a left side view of the protective vest; and FIG. 4B is a right side view of the protective vest.

FIG. 5A is a top plan view of the protective vest; and FIG. 6B is a bottom plan view of the protective vest.

FIG. 6 shows an example of eyewear, wherein the straps are shown as these may be unassembled from the lens.

FIG. 7 shows the example eyewear as it may be worn by a person, wherein stipple on the lens indicates a limited transparency material.

FIG. 8A is a front view of the eyewear; and FIG. 8B is a rear view of the eyewear.

FIG. 9A is a left side view of the eyewear; and FIG. 9B is a right side view of the eyewear.

FIG. 10A is a top view of the eyewear; and FIG. 10B is a bottom view of the eyewear.

FIG. 11 shows a rear view of the example eyewear (as in FIG. 8A), as the surrounding environment may be viewed through the lens of the eyewear.

### DETAILED DESCRIPTION

Critical incident (or traumatic event) protection (e.g., systems and devices referred to generally herein as “protective gear”) is disclosed as it may be provided for ready access and use in the event of an episode such as a school shooting, office shooting, or other event.

In an example, the protective gear may include a protective vest and eyewear. The protective vest may be worn to protect the wearer in the event of a life threatening attack. For example, the protective vest may be worn to reduce or altogether prevent the wearer from harm caused by a gun or other weapon. The eyewear may be worn, typically after an attack, to limit visual exposure of the wearer (e.g., children or other person being evacuated from the building) to traumatic images (e.g., injured or killed people).

In an example, the protective gear can be made available to a broad range of users, while encouraging use in schools and other environments without unnecessarily heightening anxiety of the occupants. For example, the protective vest may simply look like re-usable grocery bags hanging on or near a door or in a desk. The appearance of the protective vest is unlikely to raise anxiety of a potential attack like teachers wearing traditional bullet-proof vests might raise concern. Likewise, the eyewear may not appear to be out-of-place, as other eye protection is often provided in school, for example in classroom laboratories and gymnasiums

Before continuing, it is noted that as used herein, the terms “includes” and “including” mean, but is not limited to, “includes” or “including” and “includes at least” or “including at least.” The term “based on” means “based on” and “based at least in part on.”

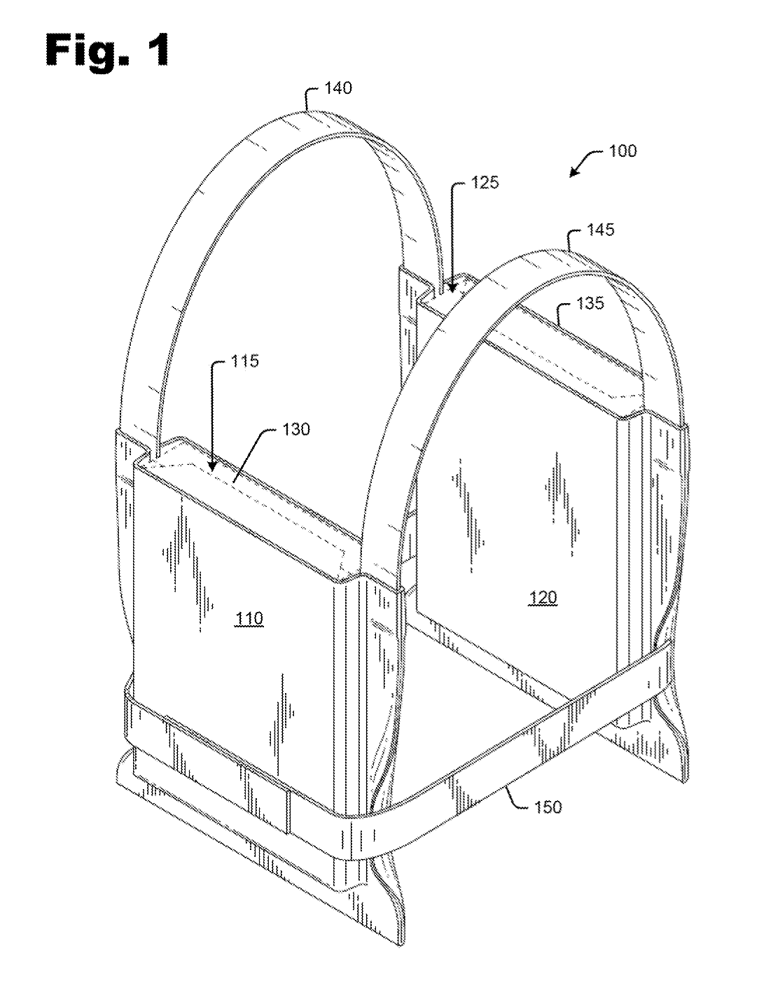

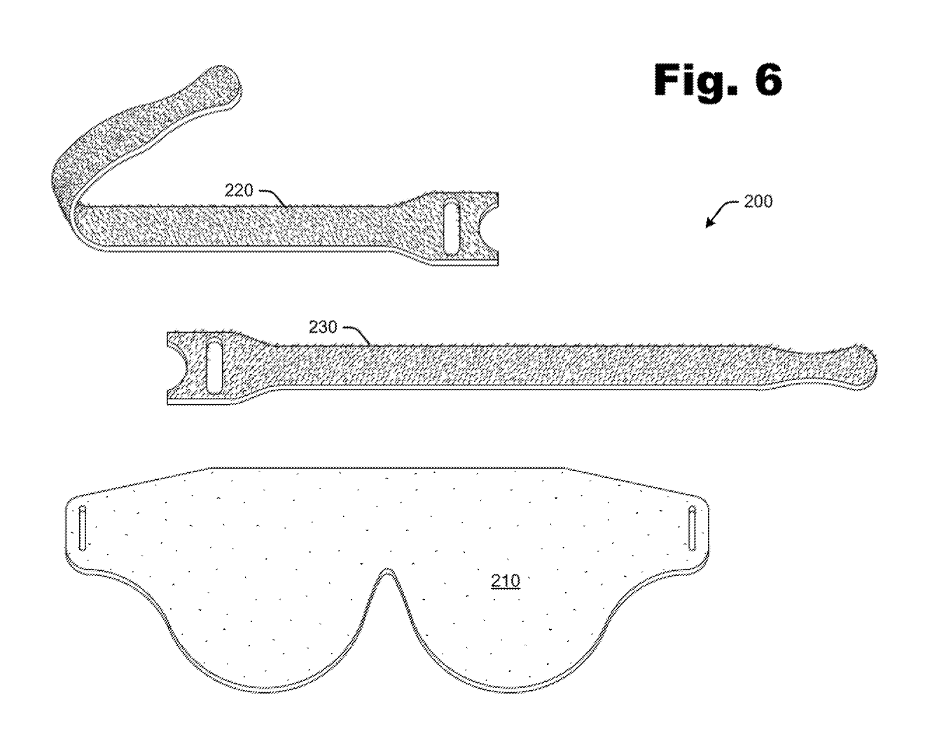

In an example, a protective system may include a protective vest 100 (illustrated in FIGS. 1-5B) and eyewear 200 (illustrated in FIGS. 6-11). It is noted that the protective system may include one or both of the protective vest 100 and the eyewear 200. it is also noted that the protective system may be provided (e.g., to a school or office environment) for different users of the same protective system. For example, the protective vest 100 may be donned by a teacher during a school shooting, because the teacher may be a likelier target than the students (e.g., having to stand guard by the door while the children hide under their desks). In this example, the eyewear 200 may be donned by the children during and/or after the shooting has occurred. The teacher may not wear the eyewear, for example, if the teacher is leading the children out of the school room.

The specific application of the protective system may vary depending on circumstances. For example, the protective vest 100 and eyewear 200 may be provided together as a package for individual users, so that each student receives or has access to the protective vest 100 and eyewear 200. Even the teacher may wear the eyewear 200, e.g., where a police officer or other emergency responder leads the entire classroom out of the building. Still other example application-scenarios are also contemplated.

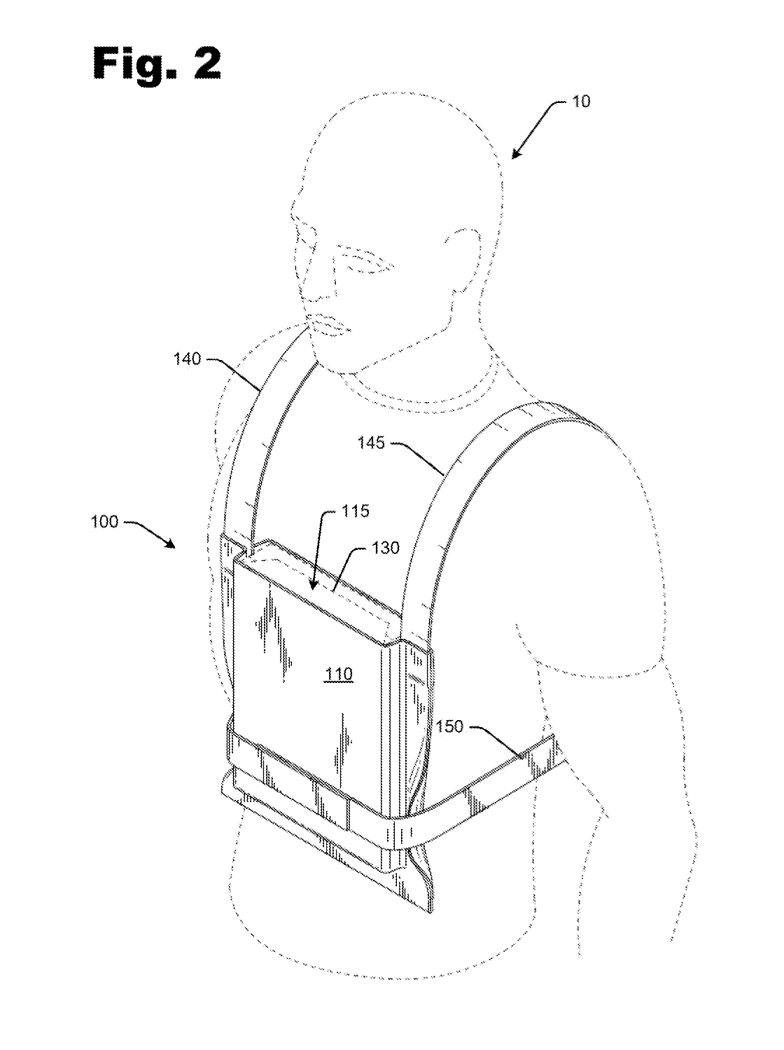

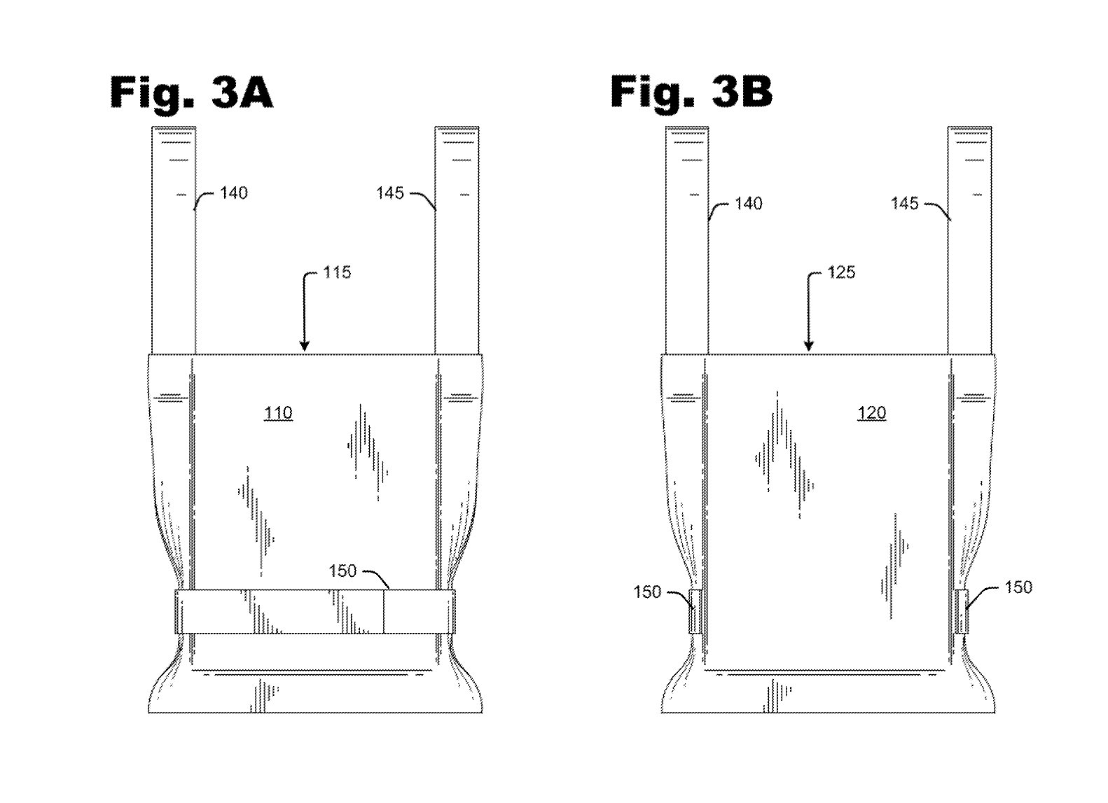

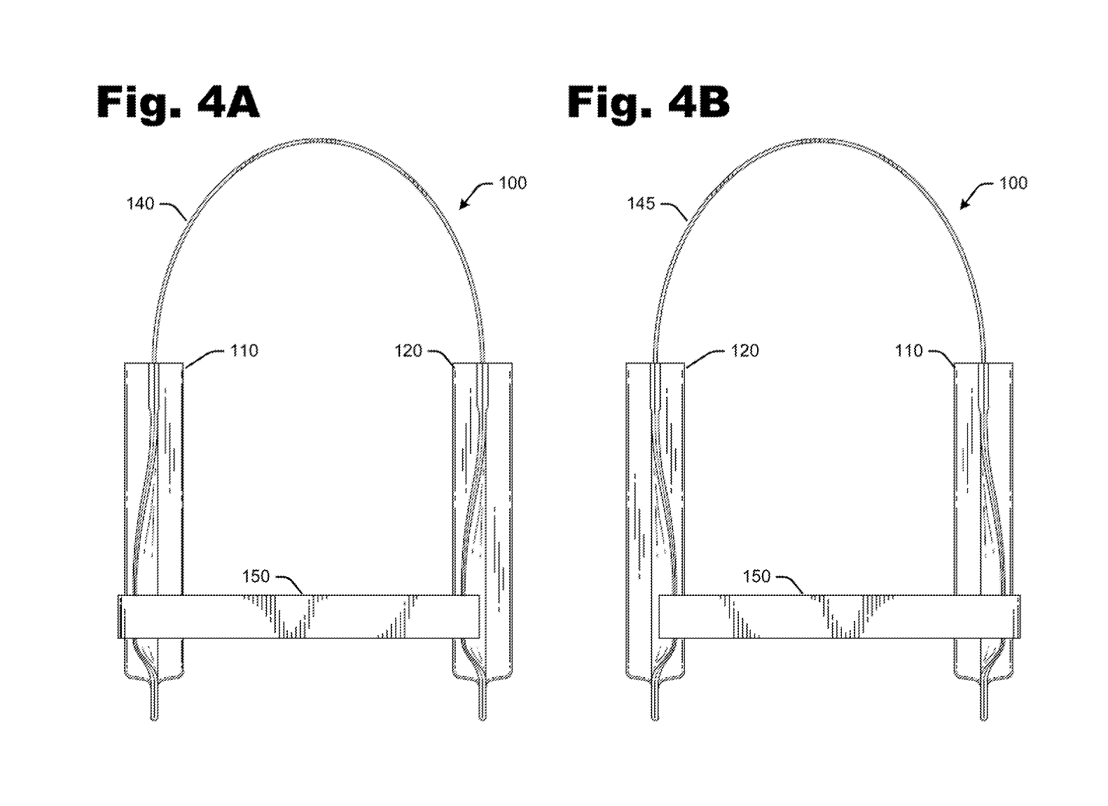

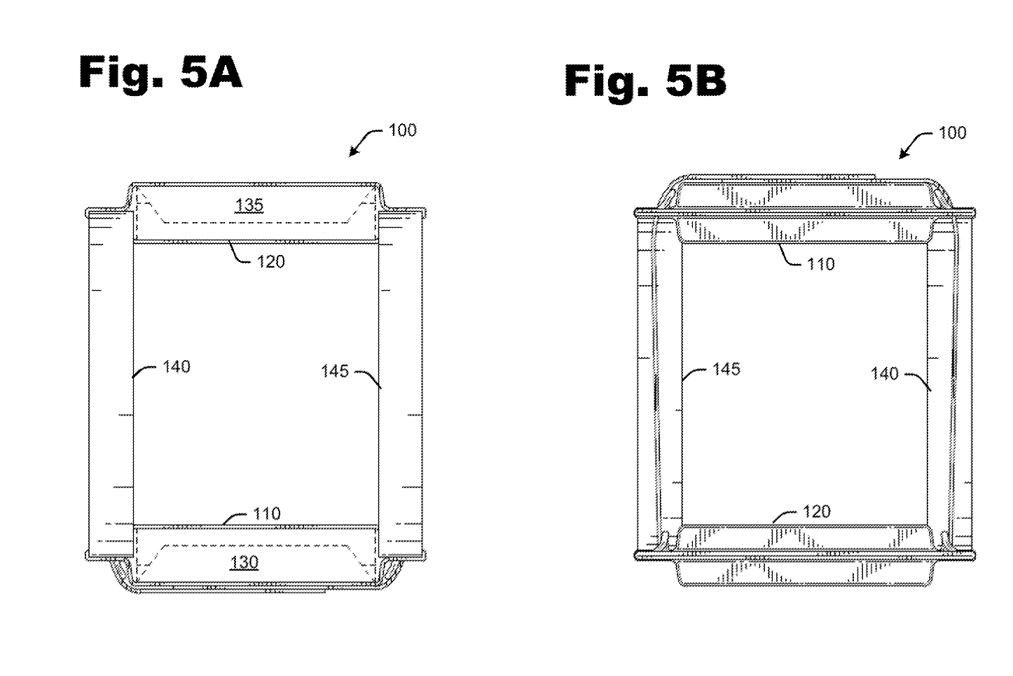

FIG. 1 is a perspective view of an example protective vest 100 shown with a ballistic barrier inserted therein. FIG. 2 is a perspective view of the example protective vest 100 shown in FIG. 1, as the protective vest 100 may be worn by a person 10. FIG. 3A is a front plan view of the protective vest 100; and FIG. 3B is a rear plan view of the protective vest 100. FIG. 4A is a left side view of the protective vest 100, and FIG. 48 is a right side view of the protective vest 100. FIG. 5A is a top plan view of the protective vest 100; and FIG. 5B is a bottom plan view of the protective vest 100.

The protective system may include a protective vest 100 to be worn by a user to protect the user from physical harm during an attack. The protective vest 100 is innocuous in design and appearance, allowing it to be “hidden in plain view,” while being available for immediate use if needed to protect both the front and rear torso of the wearer. In an example, the protective vest 100 is configured to block objects (e.g., a bullet fired from a gun or other missiles, a knife, and other penetrating and/or blunt objects that may be used as a weapon during an attack). The protective vest 100 may increase survivability of an incident by providing a protective layer to the wearer to a violent attach, such as a shooting (e.g., handgun, shotgun, rifle) or attack with other weapon or device used as a weapon.

In an example, the protective vest 100 has at least one of a front pocket 110 (or bag) and a rear pocket 120 for bag). Each front and back side pocket 110, 120 is shown to be approximately rectangular in shape, but may be fabricated in other shapes. In addition, the pockets 110, 120 of protective vest 100 can be made in a variety of sizes to fit a wide range of users.

In an example, the front pocket 110 and the rear pocket 120 are configured with openings 115, 125, respectively, to removably receive an insert or protective element 130, 135 therein. That is, the protective elements 130, 135 may be inserted into and removed from the pockets 110, 120 and are not permanently affixed therein. In an example, pockets 110, 120 may include closures, such as hook-and-loop or zipper closures. A flap or pocket cover may also be provided.

The protective element 130, 135 may include any, suitable barrier. In an example, the protective element 130, 135 is a ballistic barrier (i.e., suitable for stopping bullets from penetrating the barrier). Ballistic barriers may include material used in so-called “bullet-proof” vests worn by law enforcement and military personnel.

The protective element 130, 135 may be an approximate size of a notebook, being about 9 inches by about 12 inches, which would slide easily into a backpack sized device. Various sizes can be envisioned for smaller or larger users.

In an example, the protective element 130, 135 is a ream of paper. Paper reams (e.g., 20 pound ream) are readily available in most school and office environments and have been shown to effectively stop penetration of a bullet fired from a gun at close range. In addition, the ream of paper can be quickly and readily inserted into the pockets 110, 120 at any time prior to use.

In an example, to help keep the costs low and the effectiveness high for ballistic protection, the protective element 130, 135 (e.g., ream of paper) may be supplemented, e.g., with thin plates of steel, ceramic, steel mesh, and/or a combinations of these or other suitable materials.

Bullet-resistant materials, also called ballistic materials, or anti-ballistic materials are usually rigid, but may be supple. These materials may be complex, such as Kevlar®, Lexan®, and carbon fiber composite materials, or they may be basic and simple, such as steel or titanium. Kevlar® is a para-amid synthetic fiber material and Lexan® is a polycarbonate resin thermoplastic One may envision any number of lightweight materials, known or unknown at this time, to be used in this application including but not limited to those aforementioned or others such as ceramics, fiberglass, polymers, or composites.

It is noted that the protective element 130, 135 does not need to be provided in the protective vest 100 at all times. As such, the protective vest 100 can be folded and stored in a drawer or filing cabinet, or hang on the back of a door, enabling the protective vest 100 to be stored but still readily available for use in the event it is needed. In addition, while hanging on the back of a door or on a coat rack (or other location), the protective vest 100 has the appearance of a simple bag (e.g., a reusable grocery bag) and does not alarm those who might see the protective vest 100 to believe there is some immediate danger.

In an example, the protective vest 100 includes at least one shoulder strap (two straps 140, 145 are shown). The shoulder strap 140, 145 may serve multiple functions. For example, the shoulder strap 140, 145 may hang the protective vest 100 (e.g. on a coat rack or hook on the back of a door). The shoulder strap 140, 145 may also enable the user to wear the protective vest 100 (e.g., as shown in FIG. 2).

The shoulder strap 140, 145 may be configured as a one-size-fits-all, or be adjustable. The shoulder strap 140, 145 may also be made of any suitable material. In an example, the shoulder strap 140, 145 is made of an elastic material to enable a tighter fit on the user wearing the protective vest 100.

In an example, the protective vest 100 includes at least one waist strap 150. In an example, the waist strap 150 is a single strap that can be wrapped around the user's waist and connected at a single connection point (e.g., by hook-and-loop or other fasteners on the front of the protective vest 100). In another example, the waist strap 150 may be two separate straps (e.g., attached on each side of the rear panel of the protective vest 100 and connected together or to separate fasteners on the front panel of the protective vest 100.

During use, the user may slide his/her arms through the straps 140, 145. In an example, the user desiring to wear the vest in advance of an emergency situation, may wear both the front and back side on his or her back so as not to impede them in their normal routine. In the event of an emergency the front side can be quickly and easily lifted over the head and fitted over the torso thereby protecting the chest area.

In any event, prior to needing protection, the user secures the first or front part 110 of the protective vest 100 to hang over his or her chest, and secures the second or back part 120 to hang over his or her back. Securement may be aided by straps 140, 145, and/or 150.

The user may choose from a range of bullet-proof materials to insert into the pocket(s) 110, 120, depending on his/her risk and comfort level. For example, a user in a low-risk environment may wear a lighter material that may be less protective, whereas a user in a high risk environment may choose a heavier more protective material.

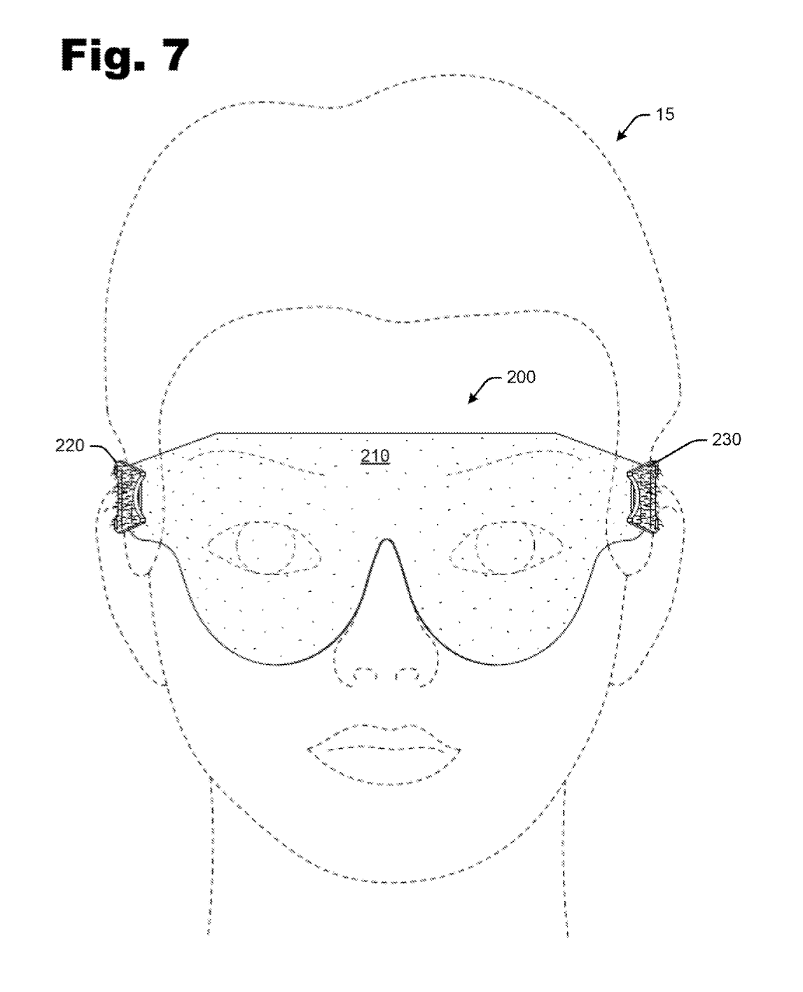

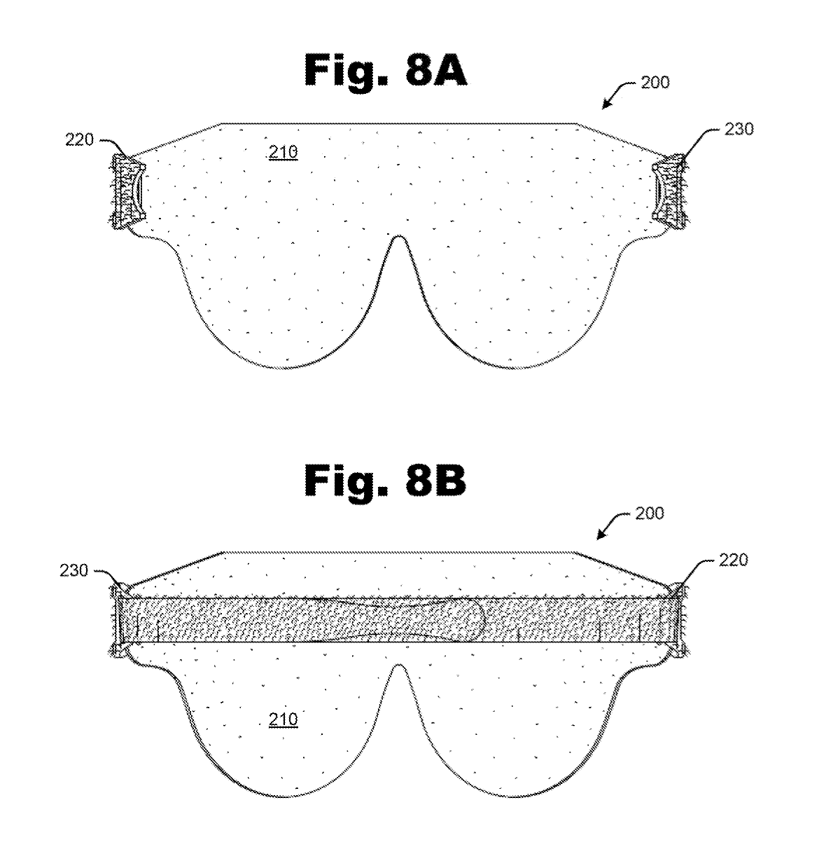

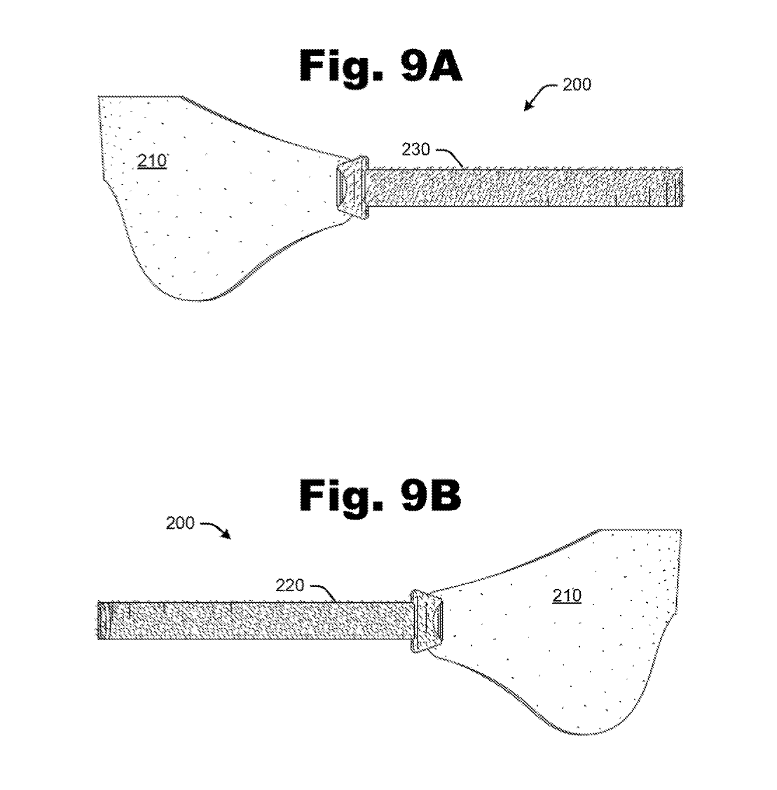

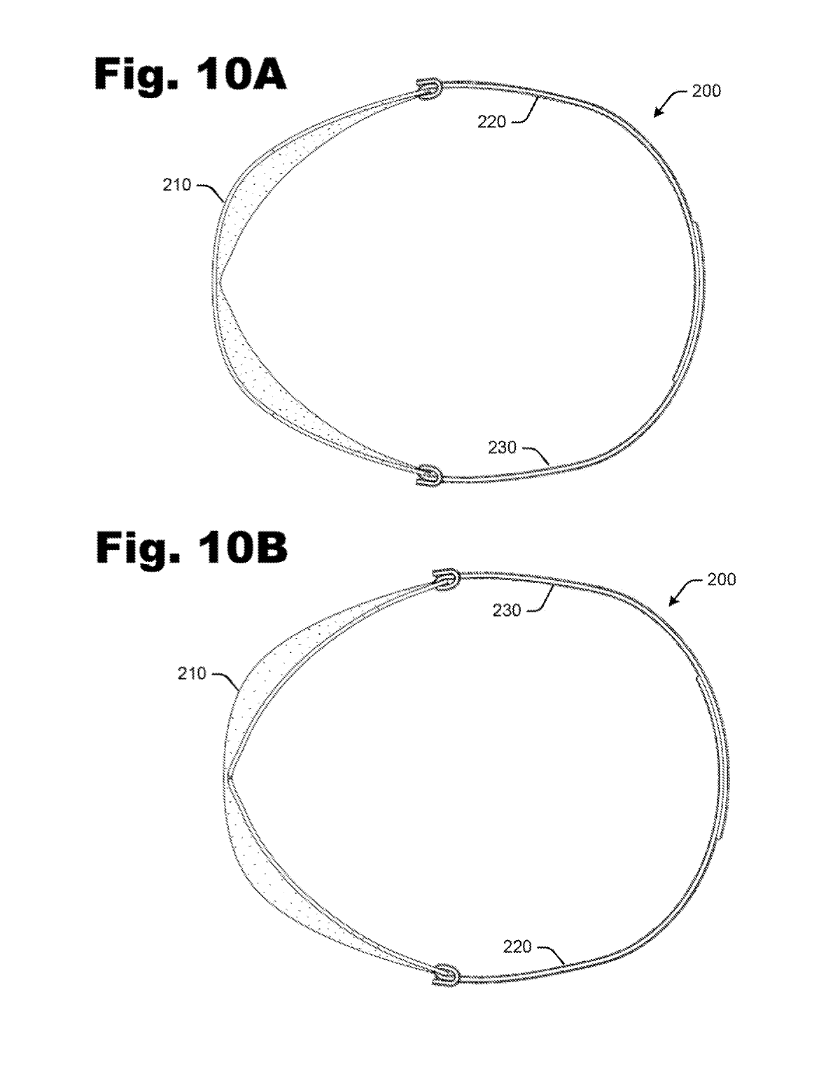

FIG. 6 shows an example of eyewear, wherein the straps are shown as these may be unassembled from the lens. FIG. 7 shows the example eyewear as it may be worn by a person, wherein stipple on the lens indicates a limited transparency material. FIG. 8A is a front view of the eyewear; and FIG. 88 is a rear view of the eyewear. FIG. 9A is a left side view of the eyewear; and FIG. 9B is a right side view of the eyewear. FIG. 10A is a top view of the eyewear; and FIG. 10B is a bottom view of the eyewear.

The protective system may include eyewear 200 to be worn by a user to protect the user from physical harm during an attack. In an example, the eyewear 200 is configured to obscure the wearer's vision (e.g., blurring or otherwise removing detail of potentially traumatic scenes), while still enabling the user to see sufficiently to move around without bumping into the wall, other people, and/or objects. The eyewear 200 may be made of an opaque silicone material with hook-and-loop or VELCRO® straps to enable the user to quickly don the eyewear and shield the user's vision from anything more than a few feet away with any clarity. The opaque color lets light transfer to the user's eyes, thereby preventing a black-out or blindfold condition, while successfully removing any clarity or sharpness from the user's field of vision. As such, the eyewear 200 may aid in restricting visual acuity and reception of harmful visual stimuli, mitigating and reducing the totality and severity of future long-term mental health conditions related to the trauma of the event (e.g., Post Traumatic Stress Disorder or PTSD).

In an example, the eyewear 200 has at least one lens 210. It is noted that the lens 210 may be a single lens (e.g., as shown in FIG. 6) or separate lenses (e.g., one lens for each eye).

In an example, the lens frame may be contoured to at least generally fit the shape of a person's face. The frame may also include one or two openings that may be round, oval, or other suitable shape to receive a strap.

The eyewear 200 may also include one or more strap. Two straps 220 and 230 are shown in FIG. 6. The straps 220 and 230 may be connected together (e.g., via hook-and-loop or other fastener such as a buckle), as illustrated in FIG. 8B. In another example, a single strap may be provided.

The strap(s) may be made of any suitable material, and in an example, the strap(s) are made of an elastic or stretchable material to assist in size adjustment and securement to the user's head 15. Again, as noted above, the user may be the same user as that of the protective vest 100 or a different user.

The lens 210 may be formed of a limited transparency material. As such, the lens 210 obscures objects viewed through the eyewear 210, such that only shadows or outlines of the objects are viewable through the eyewear.

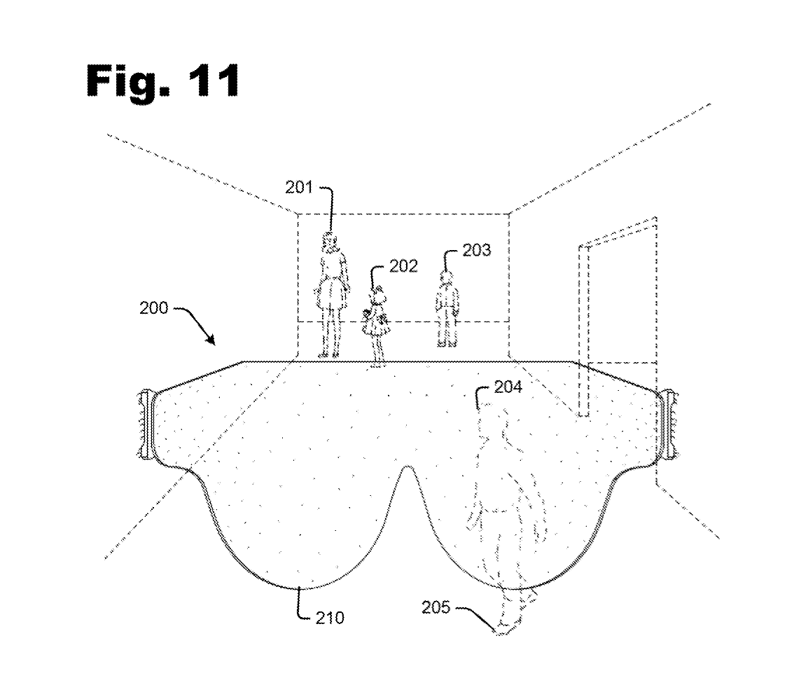

FIG. 11 shows a rear view of the example eyewear (as in FIG. 8A), as the surrounding environment may be viewed by a user through the lens 210 of the eyewear 200. It is noted that the environment seen through the lens 210 of the eyewear 210 is obscured or blurred relative to the detail of the environment seen outside of the lens of the eyewear. For example, the people 201 shown outside of the lens 210 are more clearly visible (as illustrated by the darker outline of the bodies), than the portion 204 of the person 205 within the field of view of the lens 210.

It is noted that the wearer of the eyewear 200 would only see what is in the field of view of the lens 210, as the lens 210 would be secured on the user's face close to the user's eyes. The people 201-203 and bottom portion of person 205 are only shown to be more clear in this example to illustrate the blurring effect of the lens 210.

Following a traumatic event, a person may wear the eyewear 200 over his/her eyes. The eyewear 200 allows the user to see light and dark as well as large objects and movement, but not details of a scene such as the scene shown in FIG. 11. As such, the eyewear 200 protects the user from viewing scenes that may be violent or traumatic (e.g., children or teachers injured during a school shooting) while being evacuated after a school shooting and having to walk past these injured people.

The protective gear described herein is inexpensive to make, and can be made available especially to persons who may be at high risk of viewing a traumatic incident, to protect the wearer from viewing details of a trauma. It is noted, however, that the protective gear is not limited to any end-user, and may also be used in other circumstances. For example, first responders may have the eyewear 200 available for use at an accident scene, or where the police enter a home where there has been a shooting and need to remove children from the home. Likewise, the protective vest 100 may be available to anyone who desires to have a quick and effective protection mechanism available (e.g., it can be carried in a briefcase or purse and a ream of paper added from the office, without having to alert co-workers to its presence).

It is noted that the examples shown and described are provided for purposes of illustration and are not intended to be limiting. Still other examples are also contemplated.

1. A protective system comprising:
a protective vest to be worn by a user to protect the user from physical harm during an attack; and
eyewear.

2. The protective system of claim 1, wherein the protective vest has at least one of a front pocket and a rear pocket, the front pocket and the rear pocket are configured with openings to removably receive a protective element therein.

3. The protective system of claim 2, wherein the protective element is a ballistic barrier.

4. The protective system of claim 2, wherein the protective element is a ream of paper.

5. The protective system of claim 1, wherein the protective vest has two shoulder straps.

6. The protective system of claim 1, wherein the protective vest has at least one waist strap.

7. The protective system of claim 1, wherein the eyewear is configured to obscure vision while wearing the eyewear.

8. The protective system of claim 1, wherein the eyewear has lenses formed of a limited transparency material.

9. The protective system of claim 1, wherein the eyewear obscures objects viewed through lenses of the eyewear such that only shadows or outlines of the objects are viewable through the eyewear.

10. A protective system for users during and after an attack, comprising:
a protective vest configured to block objects used during an attack; and
eyewear to obscure vision of a person wearing the eyewear after the attack.

11. The protective system of claim 10, wherein the protective vest has at least one of a front bag portion and a rear bag portion, the bag portions configured with openings to removably receive a protective element therein.

12. The protective system of claim 11, wherein the protective element is a ballistic barrier.

13. The protective system of claim 11, wherein the protective element is a ream of paper.

14. The protective system of claim 10, wherein the protective vest has at least one of a shoulder strap and a waist strap.

15. The protective system of claim 1 wherein the eyewear has a limited transparency lens.

16. The protective system of claim 10, wherein the eyewear obscures objects viewed through lenses of the eyewear such that only shadows or outlines of the objects viewed through the eyewear.

17. The protective system of claim 10, wherein a lens of the eyewear is scratched plastic.

18. The protective system of claim 10, wherein a lens of the eyewear is semi-transparent fabric.

19. A method of protecting users both during and after an attack, comprising:
providing a protective vest to be worn by a first user during the attack: and
providing eyewear to obscure vision of a second user from traumatic scenes following the attack.

20. The method of claim 19, further comprising:
providing at least one strap on the protective vest;
providing a front bag portion and a rear bag portion on the protective vest, the bag portions configured with openings to removably receive a protective element therein:
providing the eyewear with a limited transparency lens to obscure objects viewed through lenses of the eyewear such that only shadows or outlines of the objects are viewable through the eyewear.
